(19) United States
(12) Patent Application Publication
(10) Pub. No.: US 2011/0044973 A1
(43) Pub. Date: Feb. 24, 2011

(54) TREATMENT OF CYSTIC DISEASE WITH LYSOPHOSPHATIDIC ACID ANTAGONISTS

(76) Inventor: Bonnie Blazer-Yost, Indianapolis, IN (US)

Correspondence Address: BARNES & THORNBURG LLP, 11 SOUTH MERIDIAN, INDIANAPOLIS, IN, 46204, US

(21) Appl. No.: 12/766,610

(22) Filed: Apr. 23, 2010

Related U.S. Application Data
(60) Provisional application No. 61/171,914, filed on Apr. 23, 2009.

(51) Int. Cl. A61K 39/395; A61K 31/664; A61K 31/675; A61P 13/12; A61P 1/16; A61P 1/18; A61P 43/00

(52) U.S. Cl. 424/130.1; 514/119; 514/89

(57) ABSTRACT

A method for treating cystic diseases is disclosed herein. The method comprises administering lysophosphatidic acid receptor antagonists.

### CROSS REFERENCE TO RELATED APPLICATIONS

This application claims the benefit under 35 U.S.C. §119(e) of U.S. Provisional Application Ser. No. 61/171,914, filed Apr. 23, 2009, the disclosure of which is hereby incorporated herein by reference in its entirety.

### TECHNICAL FIELD

The invention described herein pertains to the treatment of cystic disease. More particularly, the invention described herein relates to methods for treating cystic disease using lysophosphatidic acid antagonists.

### BACKGROUND AND SUMMARY OF THE INVENTION

Growth of cysts may occur in various cystic diseases such as polycystic kidney disease (PKD), Bardt Biedl syndrome, nephronophthisis, Meckel Gruber syndrome and oral-facial-digital syndrome. Additionally, aberrant cyst growth may occur that is not associated with these diseases. Without being bound by theory, cyst expansion is thought to be a function of stimulation of ion secretory events that cause increased movement of electrolytes and, secondarily, water into the cyst lumen. It has been shown that in PKD renal tissue, those events may be caused by agents that increase intracellular cAMP which, in turn, stimulate ion channels in the apical plasma membrane (Ye & Grantham, N Engl J Med, 329:310-313 (1993); Grantham et al., J Clin Invest 95:195-202 (1995); Mangoo-Karim et al., Am J Physiol 269:F381-388 (1995); Davidow et al., Kidney Int 50:208-218 (1996). These observations have been extended to liver cysts formed from the cholangiocytes which line the liver bile ducts (Muchatuta et al., Exper Biol Med 234:17-27 (2009). In agreement with this contention are studies done first in rodent models of PKD and currently in clinical trials, showing that for example antagonists of the vasopressin V2 receptor in renal cells inhibit cAMP formation and cyst growth (Gattone et al. Nat Med 9:1323-1336 (2003).

Some pharmaceutical agents in clinical trials interfere with the action of hormones that increase cAMP in target cells. In the kidney, antidiuretic hormone (ADH; vasopressin) regulates salt and water balance via a signaling pathway that increases intracellular cAMP. In the bile ducts somatostatin, via a separate hormone receptor, decreases intracellular cAMP. In addition to mediating hormone action, cAMP also causes an increase in activity of the CFTR (cystic fibrosis transmembrane regulator) channel that transports Cl⁻ into the cysts with the consequent movement of fluid and cyst expansion.

Thus, agents that decrease cAMP, such as vasopressin receptor antagonists (in the kidney) or somatostatin receptor agonists (in hepatic bile ducts) are postulated to decrease cyst growth. However, these agents have shortcomings because they are organ specific. Further, these agents are problematic because they will likely also interfere with natural hormone signaling thereby leading to unwanted side effects. Thus, there is yet a need for pharmaceutical agents capable of targeting the organ systems that are affected by cystic diseases such as polycystic kidney disease, Bardt Biedl syndrome, nephronophthisis, Meckel Gruber syndrome and oral-facial-digital syndrome, as well as other cystic formations not associated with these diseases.

It has been discovered herein that treatment with LPA antagonists that block the ability of LPA to interact with its receptor may inhibit electrolyte and fluid secretion and thereby block cyst initiation and/or growth. Thus, these agents are useful in preventing cyst formation and/or expansion in the kidney and hepatic bile ducts as well as in any other organs that are normally lined with polarized epithelial cells and show cyst formation during disease progression, such as the pancreas. Therefore, LPA antagonists are useful in the treatment of cysts in tissues having such polarized epithelial cells.

### BRIEF DESCRIPTION OF THE DRAWINGS

FIG. 1 shows a comparison of Ion Transport Stimulated in Response to Human Cyst Fluid With the Response to Forskolin. Electrophysiological responses of mpkCCDc17 cells to 10% human renal cyst fluid (panel A) and forskolin (panel B). Amiloride (10 μM final concentration) was added to each set-up at time=40 minutes. Panel A is representative of five separate experiments and panel B of four experiments.

FIG. 2 shows electrophysiological responses of mpkCCDc17 cells to cyst fluid, fetal bovine serum, and lysophosphatidic acid. Similarities of the electrophysiological responses of mpkCCDc17 cells to 10% human renal cyst fluid (panel A), 10% fetal bovine serum (panel B), and 5 μM lysophosphatidic acid (panel C). Amiloride (10 μM final concentration) was added to each set-up at time=40 minutes.

FIG. 3. shows a schematic of lysophosphatidic acid (LPA) stimulated transport processes in epithelial cells in which LPA binding to the LPA receptor on the serosal side of the cell activates intracellular signaling molecules to affect the opening of electrolyte channels.

FIG. 4. shows electrophysiological recordings of the short circuit current in cellular preparations in response to administration of NASPA (10 μM).

FIG. 5. shows electrophysiological recordings of the short circuit current in cellular preparations in response to administration of VPC 32183 (10 μM).

### DETAILED DESCRIPTION

As used herein, the term “lysophosphatidic acid antagonist” generally refers to any substance that interferes with the binding and/or activity of lysophosphatidic acid at a physiological receptor. Illustratively, lysophosphatidic acid antagonists include compounds, antibodies, and the like, that bind to receptor targets of lysophosphatidic acid, including orthosteric or allosteric sites. As used herein, the term “therapeutically effective amount” generally means an amount of a lysophosphatidic acid antagonist that is capable of inhibiting cysts.

As used herein, the term “inhibiting” is understood to encompass preventing, blocking, stopping, or slowing the progression in any manner, including partially or completely reversing.

As used herein, the term “treatment of cystic disease” is understood to encompass inhibiting cyst formation or progression. It is also to be understood that the cysts may or may not be associated with polycystic kidney disease (PKD), Bardt Biedl syndrome, nephronophthisis, Meckel Gruber syndrome, or oral-facial-digital syndrome.

As used herein, the term patient generally refers to any animal, including but not limited to humans and other mammals, such as dogs, cats, cows, horses, sheep, goats, pigs, and the like.

In one illustrative embodiment, a method is described for treating cysts in a patient in need of relief, the method includes the step of administering to the patient a therapeutically effective amount of one or more lysophosphatidic acid antagonists. Illustrative of the tissue that may be treated is liver, kidney, and/or pancreas.

In one illustrative embodiment, one or more lysophosphatidic acid antagonists are administered orally.

In one illustrative embodiment, one or more lysophosphatidic acid antagonists are administered via an implanted device or included in a matrix.

In one illustrative embodiment, one or more lysophosphatidic acid antagonists are administered by injection. Methods of injecting compounds, peptides, or antibodies in formulation may be accomplished using all known techniques, including but not limited to direct infusion, subcutaneous, parenteral, or intravenous injections. The injections may also be intrahepatic, intrarenal, and intrapancreatic. Direct infusions may be made into the target area via a catheter or other temporary or permanently implanted device. Illustratively, a substance may be infused directly into the blood vessels that supply the liver, kidney, or pancreas.

In one illustrative embodiment, the methods described herein are useful for treating cysts residing in liver, kidney and/or pancreas.

In another illustrative embodiment, the methods described herein include the step of administering one or more lysophosphatidic acid antagonists to a patient with cystic disease and in need of relief.

In another illustrative embodiment one or more lysophosphatidic acid antagonists are used as a co-therapy for treating diseases that are known to ultimately result in cyst formation.

Without being bound by theory, it is believed that lysophosphatidic acid exerts physiological effects via binding to one or more of five lysophosphatidic acid receptors (LPA₁, LPA₂, LPA₃, LPA₄, and LPA₅). Lysophosphatidic acid antagonists are herein defined as molecules, including but not limited to organic compounds and peptides, which interfere with the binding of lysophosphatidic acid to a receptor. As used herein, a lysophosphatidic receptor is generally any protein molecule in a cell or on the surface of a cell to which lysophosphatidic acid can bind, causing a change in the activity of the cell. Such receptors may be coupled to G-proteins or ion channels.

Illustrative examples of lysophosphatidic acid receptor antagonists for use in the present invention include, but are not limited to, DGPP, Carbohydrate compound 14, Kil6425, L-NASPA, VPC 12204, VPC 12249, VPC 12249-10t, VPC 12249-10t-13d, VPC 32179, VPC 32183, VPC 12031, Thio-ccPA-18:1, Thio-ccPA-16:0, CHF-ccPA, Palmitoyl α-bromomethylene phosphonate, Palmitoyl α-chloromethylene phosphonate, Palmitoyl α-H2-methylene phosphonate, Palmitoyl α-OH-methylene phosphonate, Oleoyl sn-2-AO-LPA, Palmitoyl sn-2-AO-LPA, Alkoxymethylene-phosphonate-LPA (18:1), Alkoxymethylene-phosphonate-LPA (16:0), FAP-10, FAP-12, SPH, SPP, N-palmitoyl-1-serine, N-palmitoyl-1-tyrosine, 2-Amino-3-oxo-3-(tetradeeylamino)propyl dihydrogen phosphate, 2-(Acetylamino)-3-oxo-3-(tetradecylamino)propyl dihydrogen phosphate, 2-Amino-3-(octadecylamino)-3-oxopropyl dihydrogen phosphate, 1,2-(3-Octadecyloxypropane)-bis (dihydrogen phosphate), 1,2-(3-Docosanoyloxypropane)-bis(dihydrogen phosphate), Phosphoric acid monobutyl ester, Phosphoric acid monooctyl ester, Phosphoric acid monooctadecyl ester, Phosphoric acid monodocosyl ester, Acyl LPG 18:1, DPIEL, LPG 14:0, 2(s)-OMPT, 3-(N-((2-(2-((pyridin-3-ylmethylamino)carbonyl)phenyl)phenyl)carbonyl)-N-(2-(2,5-dimethoxyphenyl-)ethyl)amino)propanoic acid hydrochloride, methyl 3-(14-[4-({[1-(2-chlorophenyl)ethoxy]carbonyl}amino)-3-methyl-5-isoxazolyl]benzyl}sulfanyl)propanoate, and 4′-1[(3-phenylpropyl)(3,4,5-trimethoxybenzoyl)amino]methyl}-2-biphenylyl)acetic acid.

In one embodiment, the LPA antagonist is a cyclic sulfuric acid analog of 2-oleoyl LPA, such as described in Tamaruya et al., Angew. Chem. Int. Ed., 43: 2834-37 (2004), herein incorporated by reference.

Additional illustrative examples of lysophosphatidic acid inhibitors include antibodies that bind to one or more LPA receptors. The antibody may possess specificity for LPA₁, LPA₂, LPA₃, LPA₄, LPA₅ receptors, or any combination thereof. Antibodies directed against LPA receptors may be generated using routine methods for antibody production according to procedures well known to those skilled in the art.

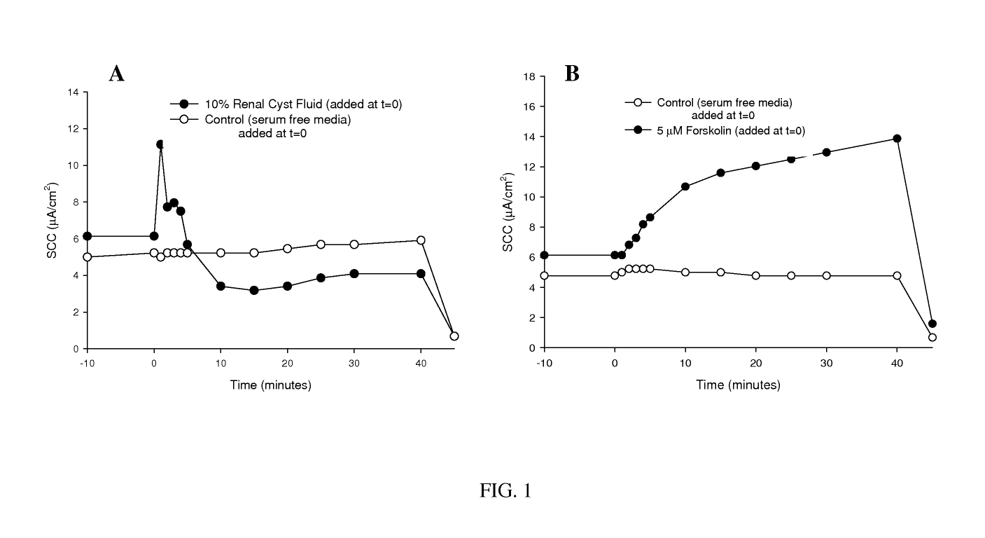

Described herein is the method of short-circuit electrophysiology used for detailed examination of the nature of the ion channels stimulated in response to cyst fluid. These studies utilized mouse principal cells of the kidney cortical collecting duct, clone 4 (mpkCCDc14) cell line. These cells have the characteristics of the principal cell type found in the distal portion of the renal nephron. Principal cells often line renal cysts. It was found that human cyst fluid stimulated Cl⁻ secretion via the cystic fibrosis transmembrane transporter (CFTR). This finding is in agreement with Granthan et al. However, the activation of other ion channels by the fluid was also discovered. When examined in real time, the nature of channel activity by the cyst was quite different than the activity seen in response to forskolin, as shown in FIG. 1. These studies indicate that there are components found in cyst fluid that stimulate multiple ion transporters, which together cause electrolyte and fluid secretion into the cyst lumen.

Extensive analyses were conducted, including a proteomic determination of the cyst fluid. Cyst fluid was separated into fractions containing components with molecular weights greater and less than 100 kDa (CENTRICON-100, AMICON). The secretory activity remained in the retentate (substances >100 kDa) indicating the factor causing the secretion is larger than forskolin (MW 0.14 kDa). It was found that the stimulatory activity appears to be due to lysophosphatidic acid (LPA) bound to a group of large molecular weight binding proteins.

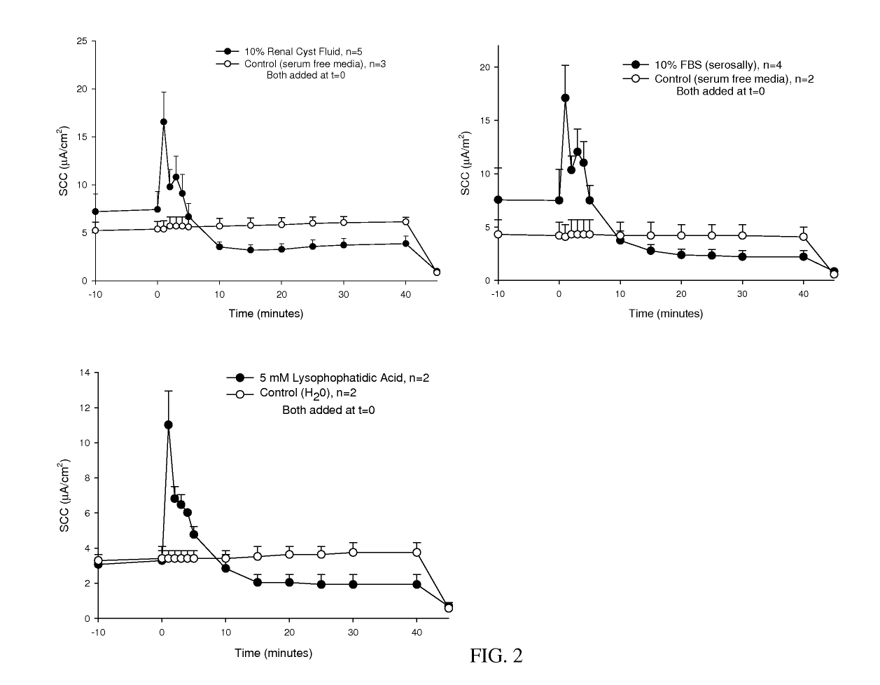

Proteomic analyses indicated most of these binding proteins were found predominately in the serum. Subsequent experiments indicated that serum, which contains 1-10 μM LPA, mimics the stimulatory activity of the cyst fluid when added directly to the normal renal cells, as shown in FIG. 2.

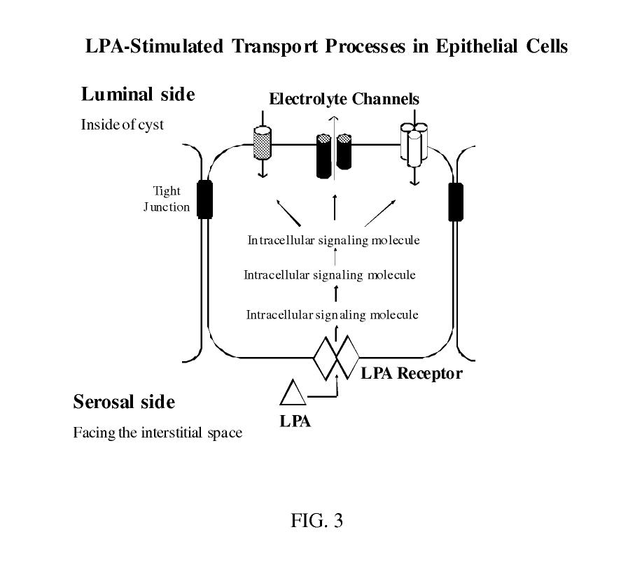

The stimulatory activity from serum, cyst fluid, purified fractions of cyst fluid or exogenous LPA, is only seen when added to the serosal side of the cells. As shown in FIG. 3, this phenomenon is analogous to adding the material to the outside of the cyst. Collectively, these data indicate that there are components from the serum that can stimulate secretory electrolyte and fluid movement. These components are not normally in direct contact with the basolateral membrane of the epithelial cells. Without being bound by theory, it is believed herein that in cystic diseases there is an increased vascular permeability that allows stimulatory serum components like LPA to directly interact with basolateral receptors on epithelial cells. These factors are not normally in direct contact with their cognate receptors on the basolateral membrane of the epithelial cells but are found in increased concentrations in the interstitial space due to a disease-related increase in vascular permeability. Without being bound by theory, it is believed that this activity causes an increase in transepithelial signaling, which stimulates ion transporters, and results in net electrolyte and fluid secretion (see, for example, FIG. 3).

Epithelial cells which line secretory or absorptive parts of organs have a polarized phenotype. When the epithelial monolayer is formed, the proteins and lipids of the apical membrane facing the luminal side are different than the proteins and lipids in the basolateral membrane facing the serosal side. The tight junctions maintain these differences.

### EXAMPLES

### Example 1

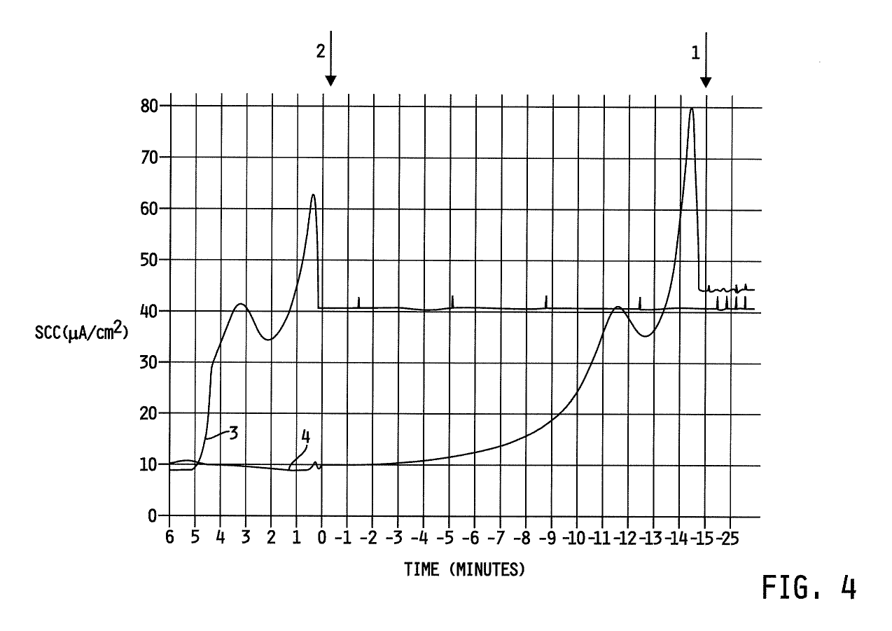

Electrophysiological testing of effects NASPA on short circuit current. Cell preparations were pre-incubated with NASPA (10 μM) or diluent (DMSO) for 15 minutes prior to administration of 10% fetal bovine serum (FBS). FBS was administered serosally at time zero. As shown FIG. 4, NASPA appeared to act like an agonist when first added. Subsequently, NASPA antagonized the LPA added as part of the fetal bovine serum.

### Example 2

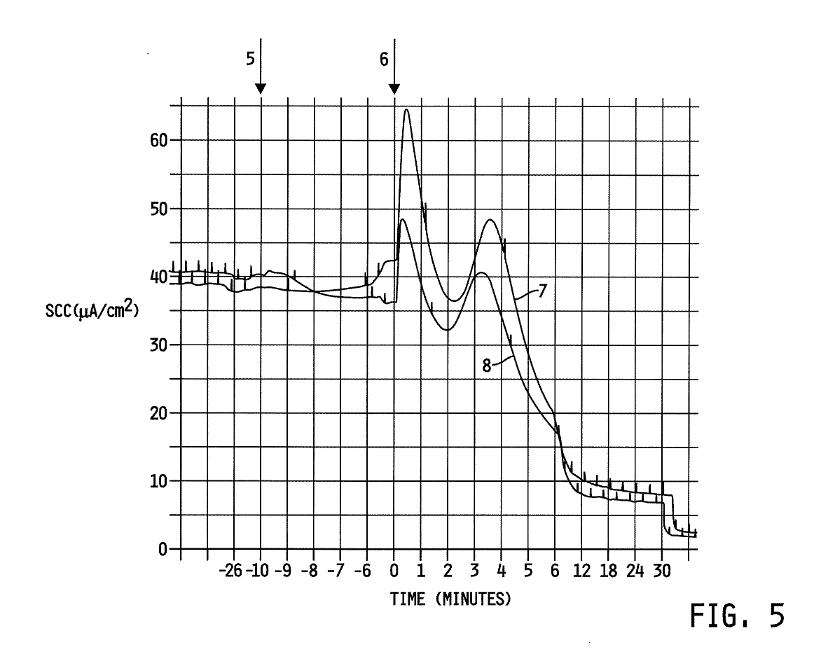

Electrophysiological testing of effects VPC 32183 on LPA induced increase in short circuit current. Cell preparations were incubated with VPC 32183 (10 μM) or diluent (3% BSA in) for 10 minutes prior to serosal administration of LPA (0.5 μM). Lysophosphatidic acid (0.5 μM in 0.50) in was added serosally at time zero. Pre-incubation with VPC 32183 (10 μM) depressed the LPA induced increases in short circuit current (SCC) as compared to diluent-treatment, as shown in FIG. 5.

### Example 3

Treatment of PCK rats with an LPA antagonist. The PCK rat expresses many of the characteristics of human ADPKD (4) but the mutation that causes the rodent form of the disease is orthologous to the human ARPKD gene, PKHD1, which encodes a protein called fibrocystin (Harric P C, Curr Opin Nephrol Hypertens 11:309-314 (2002)). These rodents develop both kidney and liver cysts and live long enough to facilitate testing of drugs post-weaning to 6-8 months of age. The PCK rat model has been used previously in tests for treatment options for PKD (Gattone et al., Nat Med 9:1323-1336 (2003); Putnam et al., J Am Soc Nephrol 18:934-43 (2007)).

PCK rats may be fed control or LPA antagonist-supplemented diets for 32 weeks starting 1 week after birth. Before weaning the animals may be fed using a liquid form of the diet delivered via pasteur pipette. After weaning the animals may be fed on the control or LPA antagonist-supplemented diet until week 32. PCK rats are bred to be homozygous for the mutation causing polycystic disease.

Animals may be routinely monitored during the entire 8 months. Alterations in the normal weight gain in the growing animals may be monitored. Lack of weight gain or weight loss would likely signify an adverse reaction to the test compound while an unusual weight gain in the test compound-treated group could indicate whole body edema. Animals may be weighed at least once a week and more often if indicated by the results.

Urines may be assessed weekly for proteinuria. Intermittent radiological imaging may be conducted to determine kidney and liver size in animals on both diets. Hematocrits and 24 hours urinary electrolytes may be measured periodically. Blood urea nitrogen (BUN) may be determined periodically to follow kidney function. At the end of the study the kidneys and livers can be used to assess and compare LPA antagonist effects on (1) overall animal health and survival; (2) kidney and liver weights as a function of overall body weight; (3) cystic volumes as a function of overall organ weight; (4) amount of fibrosis surrounding the renal and liver cysts; and (5) mitotic index of the epithelial cell layers surrounding the cysts.

### Example 4

Treatment of BALB/c-cpk/cpk mice with an LPA antagonist. The BALB/c-cpk mouse is a non-orthologous model of ARPKD that exhibits both fast (heterozygous, cpk/cpk) and slow (homozygous, cpk/+) cystic development. In the BALB/c-cpk mouse model, the mutation causing the disease resides in a protein, cystin, and these animals have a different phenotype according to the gene dose. The homozygous animals rapidly develop polycystic kidney disease with the expression of multi-organ phenotype and die within 2-4 weeks. The heterozygous animals live to breeding age and after 12-18 months express large biliary cysts which are similar to those seen in children with ARPKD. In this model, the genetic cause of the disease is different from the PCK rat model and is, therefore, an alternative model of ARPDK. Further, these animals exhibit renal, hepatic, and pancreatic disease.

BALB/c-cpk/cpk homozygous mice may be fed control or LPA antagonist-supplemented diet starting at 5 days after birth. These animals may be sacrificed at day 17 because they normally die of renal failure by the third week. The compounds may be delivered in a liquid diet. Studies indicate that the animals will readily consume a compound fed to them via a pasteur pipette. BALB/c-cpk/+ heterozygous mice may be fed control or LPA antagonist-supplemented diet starting 5 days after birth and continued post weaning for 12 months.

The BALB/c-cpk/+ heterozygous animals have a relatively normal lifespan and begin to develop cysts, predominately hepatic bile duct cysts, at 6-8 months of age. Therefore, the heterozygous animals may be placed on antagonist-supplemented diets directly after weaning and may be monitored for 12 months.

In the BALB/c-cpk/cpk mice, BUN may be assayed on blood drawn at the time of sacrifice to determine renal function. The animals may be used to compare the effects of LPA antagonist on (1) kidney, liver, and pancreas weights as a function of overall body weight; (2) cystic volumes as a function of overall organ weight; and (3) amount of fibrosis surrounding the renal, liver, and pancreatic cysts.

In the BALB/c-cpk/+ heterozygous mice urines may be assessed weekly for proteinuria. Intermittent radiological imaging may be conducted to determine kidney, liver, and pancreas size. Hematocrits and 24 hours urinary electrolytes may be measured periodically during the study. Blood urea nitrogen may be assessed periodically to follow kidney function. These animals may be used to compare the effects of an LPA antagonist on: (1) overall animal health and survival; (2) kidney, liver, and pancreas weights as a function of overall body weight; (3) cystic volumes as a function of overall organ weight; (4) amount of fibrosis surrounding the renal, liver, and pancreatic cysts; and (5) mitotic index of the epithelial cell layers surrounding the cysts.

### Example 5

Treatment of WPK rats with an LPA antagonist. The WPK rat is an orthologous, rapidly progressing, model of Meckel Gruber Syndrome. It is a rapidly progressing disease with both liver and kidney involvement. Thus, the use of an alternative, non-PKD model may facilitate a determination of whether LPA antagonists can inhibit several forms of ion-driven cystic disease.

WPK rats may be fed control or LPA antagonist-supplemented diets starting at 6 days after birth. These animals may be sacrificed at day 21 because they normally die of renal failure by the third week. The compounds may be delivered in a liquid diet. Studies indicate that the animals readily consume compounds fed to them via a pasteur pipette. Radiological determination as well as routine urine and blood chemistries may be conducted to document disease progression. At the end of each study, assessments made be made regarding (1) kidney and liver weights as a function of overall body weight; (2) cystic volumes as a function of overall organ weight; (3) amount of fibrosis surrounding the renal and liver cysts; and (4) mitotic index of the epithelial cell layers surrounding the cysts.

Each of rodent models described herein arise from separate mutations and express different time courses of disease progression. These models are useful for determining efficacy of drugs that can be used to treat cystic diseases that involve the activation of ion channels for cyst growth, including but not limited to ARPDK and ADPKD.

It is also possible to assess the efficacy of compounds that affect LPA binding and/or LPA mediated signaling in cases of very severe disease (BALB/c-cpk-cpk), and WPK where changes can be monitored during rapid progression to premature death from renal failure. The PCK and BALB/c-cpk/+ models additionally provide the ability to assess the effects of compounds that affect LPA on both kidney and liver cyst growth during slowly progressing disease.

### Example 6

Treatment of Pkhd1 knockout mice with an LPA antagonist. Mutations in PKHD1 cause autosomal recessive polycystic kidney disease. The Pkhd1 knockout mouse is an animal model for cystic disease. In the Pkhd1^{lacZ/lacZ} mouse, for example, exons 1-3 of the Pkhd1 gene are deleted and replaced with a lacZ reporter. These mice develop cysts in the pancreas and gall bladder, as well as the kidney.

Pkhd1 knockout mice may be fed with control or LPA antagonist-supplemented diets from day 5 after birth. The compounds may be delivered in a liquid diet. These animals can survive for several months, having cysts that begin to form within the first 2 months of life.

Therefore, the homozygous animals may be placed on antagonist-supplemented diets directly after weaning and may be monitored for 12 months.

In the Pkhd1 knockout mice, BUN may be assayed on blood drawn at the time of sacrifice to determine renal function. The animals may be used to compare the effects of LPA antagonist on (1) kidney, liver, and pancreas weights as a function of overall body weight; (2) cystic volumes as a function of overall organ weight; and (3) amount of fibrosis surrounding the renal, liver, and pancreatic cysts.

In the Pkhd1 knockout mice, urines may be assessed weekly for proteinuria. Intermittent radiological imaging may be conducted to determine kidney, liver, and pancreas size. Hematocrits and 24 hours urinary electrolytes may be measured periodically during the study. BUN may be assessed periodically to follow kidney function. These animals may be used to compare the effects of an LPA antagonist on: (1) overall animal health and survival; (2) kidney, liver, and pancreas weights as a function of overall body weight; (3) cystic volumes as a function of overall organ weight; (4) amount of fibrosis surrounding the renal, liver, and pancreatic cysts; and (5) mitotic index of the epithelial cell layers surrounding the cysts.

The following publications are incorporated herein in their entirety by reference:
1. Ye M, Grantham J J. The secretion of fluid by renal cysts from patients with autosomal dominant polycystic kidney disease. N Engl J Med 329:310-313, 1993
2. Grantham J J, Ye M, Gattone V H, 2nd, Sullivan L P. In vitro fluid secretion by epithelium from polycystic kidneys. J Clin Invest 95:195-202, 1995.
3. Mangoo-Karim R, Ye M, Wallace D P, Grantham J J, Sullivan L P. Anion secretion drives fluid secretion by monolayers of cultured human polycystic cells. Am J Physiol 269:F381-388, 1995.
4. Davidow C J, Maser R L, Rome L A, Calvet J P, Grantham J J. The cystic fibrosis transmembrane conductance regulator mediates transepithelial fluid secretion by human autosomal dominant polycystic kidney disease epithelium in vitro. Kidney Int 50:208-218, 1996.
5. Muchatuta M, Gattone V, Witzmann F, Blazer-Yost B L. Structural and Functional Analysis of Liver Cysts from the BALB/c-cpk Mouse Model of PKD. Exper. Biol. Med. 234:17-27, 2009.
6. Gattone V H, Wang X, Harris P C, Torres V E. Inhibition of renal cystic disease development and progression by a vasopressin V2 receptor antagonist. Nat Med 9:1323-1336, 2003.
7. Putnam W C, Swenson S M, Reif G A, Wallace D P, Helmkamp G M Jr, Grantham J J. Identification of a forskolin-like molecule in human renal cysts. J Am Soc Nephrol. 18:934-43, 2007.
8. Torres V E, Harris P C. Mechanisms of Disease: autosomal dominant and recessive polycystic kidney diseases. Nat Clin Pract Nephrol 2:40-55, 2006.
9. Nachury M V, Loktev A V, Zhang Q, Westlake C J, Peranen J, Merdes A, Slusarski D C, Scheller R H, Bazan J F, Sheffield V C, Jackson P K. A Core Complex of BBS Proteins Cooperates with the GTPase Rab8 to Promote Ciliary Membrane Biogenesis. Cell 129:1201-1213, 2007.
10. Hildebrandt F, Zhou W. Nephronophthisis-associated ciliopathies. J Am Soc Nephrol 18:1855-1871, 2007.
11. Lager D J, Qian Q, Bengal R J, Ishibashi M, Torres V E. The PCK rat: A new model that resembles human autosomal dominant polycystic kidney and liver disease. Kid Internat 59:126-136, 2001
12. Harric P C. Molecular basis of polycystic kidney disease: PKD1, PKD2 and PKHD1. Curr Opin Nephrol Hypertens 11:309-314, 2002.
13. Gattone V H, Wang X, Harris P C, Torres V E. Inhibition of renal cystic disease development and progression by a vasopressin V2 receptor antagonist. Nat Med 9:1323-1336, 2003.
14. Torres V E, Wang X, Qian Q, Somolo S, Harris PC and Gattone V H. Effective treatment of an orthologous model of autosomal dominant polycystic kidney disease. Nat Med 10:363-364, 2004
15. Masyuk T V, Masyuk A I, Tones V E, Harris P C, Larusso N F. Octreotide inhibits hepatic cystogenesis in a rodent model of polycystic liver disease by reducing cholangiocyte adenosine 3′,5′-cyclic monophosphate. Gastroenterology 132:1104-1116, 2007.
16. Ricker J L, Gattone V H 2nd, Calvet J P, Rankin C A. Development of autosomal recessive polycystic kidney disease in BALB/c-cpk/cpk mice. J Am Soc Nephrol 11:1837-1847, 2000.
17. Gattone V et al., Developing renal innervation in the spontaneously hypertensive rat: evidence for a role of the sympathetic nervous system in renal damage. J Hypertens. 8::423-428, 1990.

1. A method for treating cystic disease in a patient in need of relief, the method comprising the step of administering to the patient a therapeutically effective amount of one or more lysophosphatidic acid antagonists.

2. The method of claim 1 wherein the one or more lysophosphatidic acid antagonists are selected from the group consisting of:
DGPP, cyclic sulfuric acid analogs of 2-oleoyl LPA, Kil6425, L-NASPA, VPC 12204, VPC 12249, VPC 12249-10t, VPC 12249-10t-13d, VPC 32179, VPC 32183, VPC 12031, Thio-ccPA-18:1, Thio-ccPA-16:0, CHF-ccPA, Palmitoyl α-bromomethylene phosphonate, Palmitoyl α-chloromethylene phosphonate, Palmitoyl α-H2-methylene phosphonate, Palmitoyl α-OH-methylene phosphonate, Oleoyl sn-2-AO-LPA, Palmitoyl sn-2-AO-LPA, Alkoxymethylene-phosphonate-LPA (18:1), Alkoxymethylene-phosphonate-LPA (16:0), FAP-10, FAP-12, SPH, SPP, N-palmitoyl-1-serine, N-palmitoyl-1-tyrosine, 2-Amino-3-oxo-3-(tetradeeylamino)propyl dihydrogen phosphate, 2-(Acetylamino)-3-oxo-3-(tetradecylamino)propyl dihydrogen phosphate, 2-Amino-3-(octadecylamino)-3-oxopropyl dihydrogen phosphate, 1,2-(3-Octadecyloxypropane)-bis(dihydrogen phosphate), 1,2-(3-Docosanoyloxypropane)-bis (dihydrogen phosphate), Phosphoric acid monobutyl ester, Phosphoric acid monooctyl ester, Phosphoric acid monooctadecyl ester, Phosphoric acid monodocosyl ester, Acyl LPG 18:1, DPIEL, LPG 14:0, 2(s)-OMPT, 3-(N-((2-(2-((pyridin-3-ylmethylamino)carbonyl)phenyl)phenyl)carbonyl)-N-(2-(2,5-dimethoxyphenyl-)ethyl)amino)propanoic acid hydrochloride, methyl 3-(14-[4-({[1-(2-chlorophenyl)ethoxy]carbonyl}amino)-3-methyl-5-isoxazolyl]benzyl}sulfanyl)propanoate, and 4′-1[(3-phenylpropyl)(3,4,5-trimethoxybenzoyl)amino]methyl}-2-biphenylyl)acetic acid.

3. The method of claim 2 wherein one of the lysophosphatidic acid antagonists is VPC 32183.

4. The method of claim 1 wherein at least one of the lysophosphatidic acid antagonists interferes with the activity of lysophosphatidic acid at one or more lysophosphatidic acid receptors selected from the group consisting of LPA₁, LPA₂, LPA₃, LPA₄, and LPA₅.

5. A method for treating cystic disease in a patient in need of relief, the method comprising the step of administering to the patient a therapeutically effective amount of one or more antibodies that bind to one or more lysophosphatidic acid receptors.

6. The method of claim 1 wherein the cystic disease is associated at least in part with one or more of polycystic kidney disease (PDK), Bardt Biedl syndrome, nephronophthisis, Meckel Gruber syndrome, or oral-facial-digital syndrome.

7. The method of claim 1 wherein the cyst resides in an internal organ.

8. The method of claim 7 wherein the internal organ is selected from the group consisting of kidney, liver, and pancreas.

9. The method of claim 1 wherein the lysophosphatidic acid antagonist is administered orally, parenterally, or by injection.

10. The method of claim 9 wherein the lysophosphatidic acid antagonist is administered by injection.

11. The method of claim 10 wherein the injection is intravenous.

12. The method of claim 10 wherein the injection is intrahepatic.

13. The method of claim 10 wherein the injection is intrarenal.

14. The method of claim 10 wherein the injection is intrapancreatic.

15. The method of claim 10 wherein the injection is a direct infusion.

16. The method of claim 1 wherein the lysophosphatidic acid antagonist is administered via an implanted device.
